(19) United States
(12) Patent Application Publication
(10) Pub. No.: US 2024/0077487 A1
(43) Pub. Date: Mar. 7, 2024

(54) METHOD FOR THE DETECTION OF LUNG CANCER

(71) Applicant: Belgian Volition SRL (GB/GB), Isnes (BE)

(72) Inventors: Marielle HERZOG, Isnes (GB); Jacob Vincent MICALLEF, London (GB)

(21) Appl. No.: 18/261,392

(22) PCT Filed: Jan. 13, 2022
(86) PCT No.: PCT/EP2022/050628

§ 371 Date: Jul. 13, 2023

Related U.S. Application Data
(60) Provisional application No. 63/136,873, filed on Jan. 13, 2021.

(51) Int. Cl. G01N 33/574

(52) U.S. Cl. CPC G01N 33/57473; G01N 2333/4737; G01N 2440/12

(57) ABSTRACT

The invention relates to methods and uses of a panel of biomarkers in a body fluid sample for diagnosing and/or monitoring lung cancer, in particular early stage lung cancer. The biomarkers include particular histone modifications present on cell free nucleosomes, in combination with Carcinoembryonic Antigen (CEA).

### FIELD OF THE INVENTION

The invention relates to a body fluid test method for the detection of lung cancer using a combination biomarker panel. The invention finds particular use in the detection of early stage lung cancer and therefore can be used in combination with lung cancer screening methods such as LDCT scanning, as a simple confirmation blood-test to rule-in cancer or rule-out cancer.

### BACKGROUND OF THE INVENTION

Cancer is a common disease with a high mortality. The biology of the disease is understood to involve a progression from a pre-cancerous state leading to stage I, II, Ill and eventually stage IV cancer. For the majority of cancer diseases, mortality varies greatly depending on whether the disease is detected at an early localized stage, when effective treatment options are available, or at a late stage when the disease may have spread within the organ affected or beyond when treatment is more difficult. Late stage cancer symptoms are varied including visible blood in the stool, blood in the urine, blood discharged with coughing, blood discharged from the vagina, unexplained weight loss, persistent unexplained lumps (e.g. in the breast), indigestion, difficulty in swallowing, changes to warts or moles as well as many other possible symptoms depending on the cancer type. However, most cancers diagnosed due to such symptoms will already be late stage and difficult to treat. Most cancers are symptomless at early stage or present with non-specific symptoms that do not help diagnosis. Cancer should ideally therefore be detected early using cancer tests.

The cancer with the highest mortality rate in developed countries is lung cancer. The five-year survival rate for lung cancer is >50% for cases detected when the disease is still localized within the lungs, but only 5% when the disease has spread to other organs. Unfortunately, most lung cancer cases are diagnosed when already metastatic (57%) whilst only 16% are diagnosed at an early stage. Many other cancer diseases follow a similar pattern and, for this reason, many countries have screening programs to identify individuals with cancerous or precancerous conditions. An example screening program for lung cancer involves Low-Dose Computed Tomography (LDCT) scanning.

Some cases of cancer may be detected by palpation of the body for inappropriate lumps, nodules or masses. Any such lumps may or may not be cancerous in nature and further investigation may be required to determine whether a lump is malignant or benign in nature.

However, palpation of internal organs such as the lungs, colon or pancreas is not possible and other cancer tests are necessary. Most cancer tests can be broadly categorized as either (i) a scan to visualize a nodule, mass or lump in the body, (ii) a tissue biopsy to search for abnormal cells in the target organ or (iii) a body fluid test for a substance released by the cancer or associated or surrounding tissues. All current cancer screening methods suffer from disadvantages. Scans allow visual detection of lumps or nodules but, like palpation, often fail to differentiate between malignant nodules and indolent or non-malignant (e.g. fibrous) lumps leading to poor specificity and/or overdiagnosis. Tissue biopsy involves highly invasive surgery or needle biopsies for most tissues (e.g. lung, liver, kidney, prostate). Blood and other body fluid tests are low cost and non-invasive, but rare.

Lung cancer screening by LDCT has recently been recommended as a screening test for the early detection of the disease in high risk subjects, for example long term heavy smokers. LDCT uses low dose x-rays to visualize small early lumps or nodules in the lung. However, as with other scanning methods, any lumps observed may or may not be cancerous in nature and histological confirmation of cancer by biopsy is necessary. It is reported that 95% of all positive LDCT results are false positives and that LDCT finds an abnormality that may be cancer in at least 25% of patients who do not have cancer (Lazris et al, 2019). Many nodules of unknown aetiology are found where the nodule may or may not be malignant in nature, but is too small to biopsy. False positive results may lead to unnecessary invasive procedures and nodules of unknown aetiology may lead to repeated follow-up scans to monitor lumps with repeated exposure to further x-rays. Blood tests for lung cancer detection are not used clinically primarily due to their lack of accuracy. For example, a miRNA test that detects 21% of lung cancer cases at 76% specificity has been proposed as a front-line screening test and the EarlyCDT-Lung test that detects 41% of lung cancer cases at 87% specificity is under evaluation as a primary screening test (Midthun, 2016)

Another important failing of current screening methods is low patient compliance because failure to undergo screening may lead to early death for the patient and increases the burden of expensive late stage cancer treatment for health providers. LDCT is a recent screening development but early experience indicates poor compliance, perhaps as low as 2% (Lazris et al; 2019). The reasons for this may include the need for repeat scans every 3-6 months on nodules discovered with unknown aetiology, exposing subjects to repeated x-ray doses with the potential to accelerate cancer development in what may have been otherwise slow growing nodules. The United States Preventative Services Task Force (USPSTF) has identified a need for biomarkers to accurately discriminate between benign and malignant nodules identified on LDCT scanning (Moyer, 2014). All current cancer screening methods suffer from combinations of poor accuracy, overdiagnosis, high cost, high invasiveness, exposure to x-rays and poor patient compliance.

To address the need for a simple routine cancer blood test, many blood borne biomarkers have been investigated as potential cancer tests including carcinoembryonic antigen (CEA) for CRC, alpha-fetoprotein (AFP) for liver cancer, CA125 for ovarian cancer, CA19-9 for pancreatic cancer, CA 15-3 for breast cancer and PSA for prostate cancer. However, their clinical accuracy is too low for routine diagnostic use and they are considered to be better used for patient monitoring.

Workers in the field have also investigated numerous other biomarkers for the detection of cancer including circulating cell free nucleosomes per se (Holdenrieder et al, 2001) and inflammatory molecules such as TNFα, interleukin-6 (IL-6) and interleukin-8 (IL-8) (Chadha et al, 2014).

Although it is well known that circulating levels of cell free nucleosomes per se may be elevated in a variety of cancer conditions, cell free nucleosome measurements have not been used clinically to detect cancer or for any other clinical purpose (Holdenrieder et al, 2001). A major disadvantage of measurements of cell free nucleosome per se in clinical use is that elevated levels are a non-specific indicator of cell death and have been reported for a plethora of conditions including gynaecological diseases, autoimmune diseases, inflammatory diseases, stroke, cardiac disease, sepsis, graft vs host disease trauma and following burns, surgery or exercise (Holdenrieder et al, 2005 and Holdenrieder and Stieber, 2009). Thus, measurements of elevated levels of nucleosomes per se are considered too non-specific an indicator of disease to be used in oncology.

Circulating cell free nucleosomes containing particular epigenetic signals including particular post-translational modifications, histone isoforms, modified nucleotides and non-histone chromatin proteins have also been investigated as markers of cancer (as referenced in WO2005019826, WO2013030577, WO2013030579 and WO2013084002).

Despite recent advances, very few blood test methods are used routinely during cancer screening. There is a need to develop non-invasive blood tests for individual cancers for cancer diagnosis in general, to rule cancer in or out as a potential diagnosis in symptomatic patients or as an adjunct to other cancer detection methods.

### SUMMARY OF THE INVENTION

According to a first aspect of the invention, there is provided the use of a panel of biomarkers in a body fluid sample for diagnosing and/or monitoring lung cancer, wherein the biomarkers comprise H3K27Me3, H3K36Me3 and Carcinoembryonic Antigen (CEA).

According to a further aspect of the invention, there is provided a method of diagnosing lung cancer in a patient, comprising:
detecting or measuring the level of H3K27Me3, H3K36Me3 and CEA, in a body fluid sample obtained from the patient; and
using the level detected in the body fluid sample to determine if the patient has lung cancer.

According to a further aspect of the invention, there is provided a method of assessing if a patient requires further testing for lung cancer, comprising:
detecting or measuring the level of H3K27Me3, H3K36Me3 and CEA, in a body fluid sample obtained from the patient; and
using the level detected in the body fluid sample to determine if the patient requires further testing for lung cancer.

According to a further aspect of the invention, there is provided a method of treating lung cancer in a patient, comprising;
(i) detecting or measuring the level of H3K27Me3, H3K36Me3 and CEA, in a body fluid sample obtained from the patient;
(ii) using the level detected in the body fluid sample to determine if the patient has lung cancer; and
(iii) administering a treatment to the patient if they are determined to have lung cancer in step (ii).

According to a further aspect of the invention, there is provided a kit comprising reagents to detect H3K27Me3, H3K36Me3 and CEA.

### BRIEF DESCRIPTION OF THE FIGURES

FIG. 1: A diagram showing the characteristics of a good rule-in or rule-out test. A good rule-in test gives a positive result for at least some true cancers with no, or few false positives. For example, approximately 25% of cancer cases are confirmed as true positives at “A” with 100% specificity (i.e. with no false positives). A good rule-out test gives a negative result for at least some people who do not have a cancer with no, or few false negatives. For example approximately 35% of people with no cancer are confirmed as having no cancer at “B” with 100% sensitivity (i.e. with no false negatives).

FIG. 2: A box and dot plot showing results of a panel analysis of 220 patients with no family history of lung cancer previously screened by LDCT with tissue histology confirmation. Plasma samples were assayed for a panel of analytes comprising H3K27Me3+H3K36Me3+CEA.

FIG. 3: ROC curves for a panel analysis of patients with no family history of lung cancer previously screened positive for pulmonary nodules by LDCT and found to be either malignant or benign (non-malignant) on follow-up by tissue histology. The ROC curves show the detection of all cancerous nodules vs benign nodules, the detection of early stage (0, I, II) cancerous nodules vs benign nodules and late stage cancerous nodules (III, IV) vs benign nodules. The model was able to rule-in cancer in 27% of patients with cancerous nodules with high (95%) specificity and able to rule-out 32% of patients with non-malignant nodules as not having an early stage cancer (0, I, II) with 100% specificity.

FIG. 4: ROC curves obtained for the detection of subjects with stage 0, I, II, Ill or IV lung cancer versus control subjects with no nodules using nucleosome and CEA assay results obtained in Example 2 for patients screened for pulmonary nodules by LDCT. (a) ROC curves for CEA; (b) ROC curves for the ratio H3K27Me3/H3.1; (c) ROC curves for a decision tree analysis wherein patients were classified as positive for cancer if either the CEA level was abnormal (>5 ng/ml) or if the logistic regression result for H3K27Me3/H3.1+H3K36Me3/H3.1 was abnormal.

### DETAILED DESCRIPTION OF THE INVENTION

Low-Dose Computed tomography (LDCT) is the widely accepted standard for screening of individuals at high risk of lung cancer. However, LDCT has several limitations including the high prevalence of non-malignant nodules detected leading to overdiagnosis, the potential harms of cumulative radiation dose and poor adherence to recommended follow-up. Therefore, a novel blood-based test could offer a simple follow-up confirmation approach to help to discriminate between lung cancer and non-malignant nodules. Patients would benefit from either or both of a blood-test to rule-in cancer or rule-out cancer.

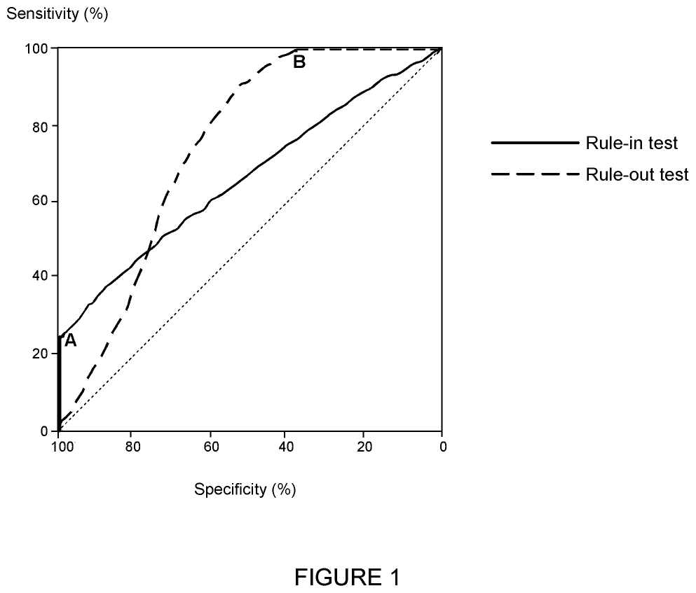

A rule-in test provides a result identifying those patients in whom there is a high probability of cancer. In the context of a patient in whom a pulmonary nodule of unknown aetiology is found by LDCT this would identify patients in whom the nodule is malignant. The characteristics of a good rule-in test therefore include a low false positive rate to provide confidence that a nodule is cancerous in nature. In terms of Receiver Operating Characteristic (ROC) curves, this corresponds to those patients identified as positive for cancer at very high clinical specificity at the bottom-left of the ROC curve as shown in FIG. 1 (i.e. those patients identified as having cancer whilst not falsely diagnosing a cancer in any patients who do not have a cancer).

A rule-out test provides a result identifying those patients in whom there is a low probability of cancer. In the context of a patient in whom a pulmonary nodule of unknown aetiology is found by LDCT this would identify patients with nodules that are highly unlikely to be a cancer and who therefore do not need treatment or aggressive invasive testing. These patients may therefore be followed up less intensively or discharged until the next scheduled screening test. The characteristics of a good rule-out test therefore include a low false negative rate to provide confidence that a nodule is not cancerous in nature. In terms of ROC curves, this corresponds to those patients identified as negative for cancer at very high clinical sensitivity at the top-right of the ROC curve as shown in FIG. 1 (i.e. those patients identified as not having cancer whilst missing nobody with a cancer).

According to a first aspect of the invention, there is provided the use of a panel of biomarkers in a body fluid sample for diagnosing and/or monitoring a lung cancer, wherein the biomarkers comprise H3K27Me3, H3K36Me3 and Carcinoembryonic Antigen (CEA).

Data is presented herein that shows that the panel of biomarkers of the invention was able to discriminate between patients with lung cancer compared to those with benign pulmonary nodules. Blood samples were obtained from patients that were referred for CT scans after pulmonary nodules were detected during LDCT screening, and tested against the panel of biomarkers. Results from the panel were able to rule out cancer in 32% of people with non-malignant nodules as not having an early stage lung cancer (i.e. stage 0, I and II).

LDCT has a very high clinical sensitivity and detects most lung cancers. The major issue for LDCT is poor clinical specificity for differentiation of small malignant and non-malignant nodules leading to unnecessary biopsy or repeat scans. Therefore a blood test would be useful: to discriminate malignant nodules; to help to choose a treatment (i.e. whether surgery is required or not); and to help to eliminate the frequency of radiation exposure from repeat/follow-up scans.

The panel of biomarkers comprises the histone post-translational modifications: H3K27Me3 (i.e. tri-methylated histone H3 at the lysine residue in position 27) and H3K36Me3 (i.e. tri-methylated histone H3 at the lysine residue in position 36). Therefore, according to one aspect of the invention, there is provided the use of a panel of biomarkers in a body fluid sample for diagnosing and/or monitoring a lung cancer, wherein the biomarkers comprise H3K27Me3 and H3K36Me3. This panel was also combined with detection of carcinoembryonic antigen (CEA) which is a blood-borne biomarker previously associated with colorectal cancer. Therefore, in a further embodiment, the panel additionally comprises CEA.

The nucleosome is the basic unit of chromatin structure and consists of a protein complex of eight highly conserved core histones (comprising of a pair of each of the histones H2A, H2B, H3, and H4). Around this complex is wrapped approximately 146 base pairs of DNA. Another histone, H1 or H5, acts as a linker and is involved in chromatin compaction. The DNA is wound around consecutive nucleosomes in a structure often said to resemble “beads on a string” and this forms the basic structure of open or euchromatin. In compacted or heterochromatin this string is coiled and super coiled into a closed and complex structure (Herranz and Esteller (2007)).

In one embodiment, the panel comprises one or more additional biomarkers. In a further embodiment, the additional biomarker is C-Reactive Protein (CRP). The level of CRP has previously been measured to detect the presence of inflammation in the body, for example due to an infection, cardiovascular disease or a chronic inflammatory disease, such as rheumatoid arthritis or lupus. Addition of CRP to the panel of biomarkers increased the specificity of the test to detect lung cancer (i.e. a rule-in test), particularly in patients who were smokers.

Additional biomarkers may also comprise cell free nucleosomes per se and one or more epigenetic features of cell free nucleosomes. References to “nucleosome” may refer to “cell free nucleosome” when detected in body fluid samples. It will be appreciated that the term “cell free nucleosome” used throughout this document is intended to include any cell free chromatin fragment that includes one or more nucleosomes. Epigenetic signal structures/features of a cell free nucleosome as referred herein may comprise, without limitation, one or more histone post-translational modifications, histone isoforms/variants, modified nucleotides and/or proteins bound to a nucleosome as a nucleosome-protein adduct. References to “nucleosomes per se” refers to the total nucleosome level or concentration present in the sample, regardless of any epigenetic features the nucleosomes may or may not include. This type of assay is also often referred to simply as a “nucleosome assay” or as a “total nucleosome assay” and typically involves detecting a histone protein common to all nucleosomes, such as histone H4 or histone H3. Therefore, in one embodiment, nucleosomes per se are measured by detecting a core histone protein, such as histone H3. As described herein, histone proteins form structural units known as nucleosomes which are used to package DNA in eukaryotic cells. In one embodiment, the histone protein is a core histone, such as H2A, H2B, H3 or H4. As previously reported in WO2016067029 (incorporated herein by reference), particular histone variants, such as histone H3.1, H3.2 or H3t, may be used to isolate cell free nucleosomes originating from tumour cells. Therefore, the level of cell free nucleosomes of tumour origin may be detected.

Total cell free nucleosomes or nucleosomes per se may also be measured by quantifying their DNA fragment content. Circulating cell free DNA (ccfDNA) in blood comprises DNA fragments of <200 base pairs in length that circulate in the form of chromatin fragments and particularly nucleosomes. It has been shown that blood measurements of ccfDNA made using the PicoGreen nucleic acid stain method correlate 95% with ELISA measurements of cell free nucleosomes (Bjorkman et al; 2003). Thus, ccfDNA measurements can be considered equivalent to, or a proxy for, measurements of total nucleosome or total chromatin fragment levels. Typical methods, without limitation, for the quantification of ccfDNA as a proxy measurement of nucleosomes include quantification using a nucleic acid stain (for example PicoGreen, SYBR Green, SYBER Gold, Oxazole yellow and Thiazole Orange) or by Polymerase Chain Reaction (PCR) methods for the amplification and measurement of a repetitive DNA sequence of a single copy gene sequence or other methods. Therefore, in one embodiment, the cell free chromatin fragment is measured (or quantified) by detecting ccfDNA. In a further embodiment, the ccfDNA is measured using a nucleic acid stain. In a further embodiment, the ccfDNA is measured by PCR. Furthermore, according to a further aspect of the invention, there is provided the use of a panel of biomarkers in a body fluid sample for diagnosing and/or monitoring a cancer, wherein the biomarkers comprise a measurement of ccfDNA.

Mononucleosomes and oligonucleosomes can be detected by Enzyme-Linked ImmunoSorbant Assay (ELISA) and several methods have been reported (Salgame et al, 1997; Holdenrieder et al, 2001; van Nieuwenhuijze et al, 2003; WO2005019826; WO2013030577; WO2013030579; and WO2013084002, all of which are herein incorporated by reference). These assays typically employ an anti-histone antibody (for example anti-H2B, anti-H3 or anti-H1, H2A, H2B, H3 and H4) as capture antibody and detection antibody (which varies depending upon the moiety to be detected) or an anti-histone antibody as capture antibody and an anti-DNA antibody as detection antibody. In one embodiment, the anti-histone antibody comprises an anti-H3 antibody or an anti-H1 antibody.

Circulating nucleosomes are not a homogeneous group of protein-nucleic acid complexes. Rather, they are a heterogeneous group of chromatin fragments originating from the digestion of chromatin on cell death and include an immense variety of epigenetic structures including particular histone isoforms (or variants), post-translational histone modifications, nucleotides or modified nucleotides, and protein adducts. It will be clear to those skilled in the art that an elevation in nucleosome levels will be associated with elevations in some circulating nucleosome subsets containing particular epigenetic signals including nucleosomes comprising particular histone isoforms (or variants), comprising particular post-translational histone modifications, comprising particular nucleotides or modified nucleotides and comprising particular protein adducts. Assays for these types of chromatin fragments are known in the art (for example, see WO2005019826, WO2013030579, WO2013030578, WO2013084002 which are herein incorporated by reference).

In one embodiment, the epigenetic feature is selected from a histone post-translational modification, histone isoform, modified nucleotide and/or a protein bound to a nucleosome (i.e. as a nucleosome-protein adduct). It will be understood that the terms “epigenetic signal structure” and “epigenetic feature” are used interchangeably herein. They refer to particular features of the nucleosome that may be detected.

In one embodiment, the cell free nucleosome comprises a histone isoform. The nucleosome moiety measured as part of the biomarker panel may be a circulating cell free nucleosome containing one or more particular or specified histone isoforms. Many histone isoforms are known in the art. The nucleotide sequences of a large number of histone isoforms are publicly available for example in the National Human Genome Research Institute NHGRI Histone DataBase (Marino-Ramirez et al. The Histone Database: an integrated resource for histones and histone fold-containing proteins. Database Vol. 2011), the GenBank (NIH genetic sequence) DataBase, the EMBL Nucleotide Sequence Database and the DNA Data Bank of Japan (DDBJ). In a preferred embodiment, the cell free nucleosome comprises a histone isoform of histone H3, for example a histone isoform selected from H3.1, H3.2 and H3t.

In another embodiment, the cell free nucleosome comprises one or more particular or specified post-translational histone modifications. The structure of nucleosomes can vary by post translational modification (PTM) of histone proteins. PTM of histone proteins typically occurs on the tails of the core histones and common modifications include acetylation, methylation or ubiquitination of lysine residues as well as methylation of arginine residues and phosphorylation of serine residues and many others. Many histone modifications are known in the art and the number is increasing as new modifications are identified (Zhao and Garcia, 2015).

In one embodiment, a group or class of related histone (post translational) modifications (rather than a single modification) is detected. A typical example of this embodiment, without limitation, would involve a 2-site immunoassay employing one antibody or other selective binder directed to bind to nucleosomes and one antibody or other selective binder directed to bind the group of histone modifications in question. Examples of such antibodies directed to bind to a group of histone modifications would include, for illustrative purposes without limitation, anti-pan-acetylation antibodies (e.g. a Pan-acetyl H4 antibody), anti-citrullination antibodies or anti-ubiquitin antibodies.

In one embodiment, the cell free nucleosome comprises one or more DNA modifications (i.e. modified nucleotides). In addition to the epigenetic signalling mediated by nucleosome histone isoform and histone post-translational modification composition, nucleosomes also differ in their nucleotide and modified nucleotide composition. Global DNA hypomethylation is a hallmark of cancer cells and some nucleosomes may comprise more 5-methylcytosine residues (or 5-hydroxymethylcytosine residues or other nucleotides or modified nucleotides) than other nucleosomes. 5-hydroxymethylation may be detected, for example, at CpG islands in the genome. In one embodiment, the DNA modification is selected from 5-methylcytosine or 5-hydroxymethylcytosine.

In another embodiment, the cell free nucleosome comprises a protein adduct, i.e. a nucleosome and another non-histone protein which is adducted to the nucleosome or chromatin fragment. Such adducts may include any protein that contains or includes a DNA binding domain or a nucleosome binding domain or a histone binding domain. Examples include transcriptions factors, structural chromatin proteins, CpG methyl-CpG binding domain proteins, high mobility group box proteins (e.g. HMGB1), epigenetic enzymes such as histone acetyl transferases, histone methyl transferases, histone deacetylases, DNA methyltransferases, PARP (poly-ADP ribose polymerase) binders and many others.

In one embodiment, the protein adducted to the nucleosome (and which therefore may be used as a biomarker) is selected from: a transcription factor, a High Mobility Group Protein or chromatin modifying enzyme. References to “transcription factor” refer to proteins that bind to DNA and regulate gene expression by promoting (i.e. activators) or suppressing (i.e. repressors) transcription. Transcription factors contain one or more DNA-binding domains (DBDs), which attach to specific sequences of DNA adjacent to the genes that they regulate. All of the circulating nucleosomes and nucleosome moieties, types or subgroups described herein may be useful in the present invention.

The data provided herein found that determining the proportion of cell free nucleosomes containing the histone markers of interest was able to improve the discrimination of the claimed biomarker panel, in particular for early stage lung cancer. Therefore, in one embodiment, the biomarkers H3K27Me3 and H3K36Me3 are measured as a ratio of the level of cell free nucleosomes or a component thereof in the sample. Cell free nucleosomes and components thereof as are defined hereinbefore. In particular, H3K27Me3 and H3K36Me3 may be measured as a ratio of the level of cell free nucleosomes containing a histone H3 variant, such as histone H3.1, in the sample.

The sample may be any biological fluid (or body fluid) sample taken from a subject including, without limitation, cerebrospinal fluid (CSF), whole blood, blood serum, plasma, menstrual blood, endometrial fluid, urine, saliva, or other bodily fluid (stool, tear fluid, synovial fluid, sputum), breath, e.g. as condensed breath, or an extract or purification therefrom, or dilution thereof. In a preferred embodiment, the body fluid sample is selected from blood, serum or plasma. Biological samples also include specimens from a live subject, or taken post-mortem. The samples can be prepared, for example where appropriate diluted or concentrated, and stored in the usual manner. It will be understood that methods and uses of the present invention find particular use in blood, serum or plasma samples obtained from a patient. In one embodiment, the sample is a blood or plasma sample. In a further embodiment, the sample is a serum sample. In a further embodiment both serum and plasma samples are used for the measurement of different members of an assay panel.

In one embodiment, the biomarkers are for use in diagnosing the stage of cancer. Cancer may be assigned as stage 0, stage I, stage II, stage III and stage IV. Stage definition varies with different cancer diseases and is known in the art. Typically, stage I is classified as when the cancer is small and confined locally to the tissue of origin. Stage II is classified as when the cancer has grown larger and beyond its origin into nearby tissues within the organ or to nearby lymph nodes. Stage III is classified as when the cancer has grown into nearby tissues beyond the organ of origin but has not spread to other more distant parts of the body. Stage IV is classified as when the cancer has spread to one or more distant parts of the body, such as the liver or lungs. Early stage cancer generally includes stages 0, I and II. Late stage cancer generally includes stages III and IV.

In one embodiment, the cancer is a stage I (for example stage IA or stage IB), stage II (for example stage IIA or stage IIB), stage III (for example stage IIIA, stage IIIB or stage IIIC) or stage IV (for example stage IVA or stage IVB) cancer. The invention may find utility in detecting early stage cancers, in particular stages I and II. Therefore, in one embodiment the cancer is a stage I, II or III. In a further embodiment, the cancer is stage I or stage II. In an alternative embodiment, the cancer is stage II or stage III. The invention may also find utility in detecting late stage cancers, in particular stages III and IV. Therefore, in one embodiment the cancer is a stage III or IV. In a further embodiment, the cancer is stage IV.

According to a further aspect of the invention, there is provided there is provided the use of H3K27Me3, H3K36Me3 and CEA binding agents in the manufacture of a kit for diagnosing and/or monitoring lung cancer in a body fluid sample.

Diagnosis methods According to a further aspect of the invention, there is provided a method of diagnosing lung cancer in a patient, comprising:
detecting or measuring H3K27Me3, H3K36Me3 and CEA, in a body fluid sample obtained from the patient; and
using the level detected in the body fluid sample to determine if the patient has lung cancer.

In another aspect, the method of the invention is performed to identify a subject at high risk of having lung cancer and therefore in need of further testing (i.e. further lung cancer investigations). The further testing may involve biopsy and one or more endoscopic or scanning methods (for example LDCT).

In addition to their uses as stand-alone tests, cancer rule-in or rule-out blood tests may be useful as adjunct methods to other screening modalities including, for example, in LDCT positive persons. LDCT positive patients have a lump or nodule in their lung but the nodule may not be malignant and LDCT has a specificity of around 60%.

In one embodiment, the lung cancer is early stage lung cancer (i.e. stage 0, I or II). In an alternative embodiment, the lung cancer is late stage lung cancer (i.e. stage III or IV).

In one embodiment, the patient has a pulmonary nodule. This may have been identified, for example, by an LDCT scan.

In one embodiment, the method additionally comprises determining at least one clinical parameter for the patient. This parameter can be used in the interpretation of results. Clinical parameters may include any relevant clinical information for example, without limitation, gender, weight, Body Mass Index (BMI), smoking status and dietary habits. Therefore, in one embodiment, the clinical parameter is selected from the group consisting of: smoking status, family history of lung cancer, age, sex and body mass index (BMI). In a further embodiment, the clinical parameter is selected from the group consisting of: smoking status and family history of lung cancer.

In one embodiment individual assay cut-off levels are used and the patient is considered positive in the panel test if individual panel assay results are above (or below if applicable) the assay cut-off level for all or a minimum number of the panel assays (for example, one of two, two of two, two of three etc). In one embodiment of the invention a decision tree model or algorithm is employed for analysis of the results.

It will be clear to those skilled in the art, that any combination of the biomarkers disclosed herein may be used in panels and algorithms for the detection of cancer and that further markers may be added to a panel including these markers.

Methods of Treatment

According to a further aspect of the invention, there is provided a method of treating lung cancer in a patient, comprising;
(i) detecting or measuring H3K27Me3, H3K36Me3 and CEA, in a body fluid sample obtained from the patient;
(ii) using the level detected in the body fluid sample to determine if the patient has lung cancer; and
(iii) administering a treatment to the patient if they are determined to have lung cancer in step (ii).

In one embodiment, the method additionally comprises performing one or more scanning methods on the subject (for example, prior to step (i) or (iii)). For example, the scanning method may be LDCT.

Treatments available for lung cancer include surgery (including biopsy), radiotherapy (including brachytherapy), hormone therapy, immunotherapy, as well as a variety of drug treatments for use in chemotherapy. In one embodiment, the treatment(s) administered are selected from: surgery, radiotherapy, hormone therapy, immunotherapy and/or chemotherapy.

According to another aspect of the invention there is provided a method of treatment for lung cancer comprising identifying a patient in need of treatment for lung cancer using a panel test of the invention and providing said treatment, wherein the panel test comprises reagents to detect H3K27Me3, H3K36Me3 and CEA, in a body fluid sample obtained from the patient.

In one embodiment, patients are at high risk for lung cancer if they have elevated levels compared to a control.

In one embodiment, the control comprises a healthy subject, a non-diseased subject and/or a subject without cancer. In one embodiment, the method comprises comparing the amount of biomarker(s) present in a body fluid sample obtained from the subject with the amount of biomarker(s) present in a body fluid sample obtained from a normal subject. It will be understood that a “normal” subject refers to a healthy/non-diseased subject.

In one embodiment, the control comprises a subject with a non-cancerous disease. Methods of the invention are able to distinguish between subjects with cancer and subjects with a non-malignant nodule. Therefore, in one aspect the diagnosis comprises differential diagnosis of a patient with lung cancer from a patient with a non-malignant (i.e. benign) nodule.

Methods of Patient Assessment

The invention finds particular use in assessing whether a patient requires further investigation for the cancer (e.g. for diagnosis and/or identification of organ location). Such procedures, including LDCT scans, other scans and biopsies are invasive or potentially hazardous and are relatively costly to healthcare providers. Therefore there is a need to reduce the number of patients sent for unnecessary investigations. For example, this aspect of the invention will be useful for assessing LDCT positive persons as in need of a biopsy. Therefore, according to a further aspect of the invention, there is provided a method of assessing if a patient requires further testing for lung cancer, comprising:
detecting or measuring the level of H3K27Me3, H3K36Me3 and CEA, in a body fluid sample obtained from the patient; and
using the level detected in the body fluid sample to determine if the patient requires further testing for lung cancer.

In one embodiment, the further test for lung cancer is a lung biopsy.

In one embodiment, the patient has a pulmonary nodule. This may have been identified, for example, by an LDCT scan.

According to a further aspect of the invention there is provided a method of identifying a patient in need of a LDCT scan comprising applying a body fluid sample obtained from the patient to a panel test as defined herein, and using the results obtained from the panel test to identify whether the patient is in need of a scan.

In one embodiment, the method described herein is repeated on multiple occasions. This embodiment provides the advantage of allowing the detection results to be monitored over a time period. Such an arrangement will provide the benefit of monitoring or assessing the efficacy of treatment of a disease state. Such monitoring methods of the invention can be used to monitor onset, progression, stabilisation, amelioration, relapse and/or remission.

Thus, the invention also provides a method of monitoring efficacy of a therapy for a disease state in a subject, suspected of having such a disease, comprising detecting and/or quantifying the biomarker (e.g. biomarker panel described herein) present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the nature or amount of the biomarker(s) in test samples taken on different occasions.

Thus, according to a further aspect of the invention, there is provided a method for monitoring efficacy of therapy for a disease state in a human or animal subject, comprising:
(a) quantifying the panel biomarkers as defined herein; and
(b) comparing the panel result in a test sample with that of one or more control(s) and/or one or more previous test sample(s) taken at an earlier time from the same test subject.

A change in the biomarker result in the test sample relative to the level in a previous test sample taken earlier from the same test subject may be indicative of a beneficial effect, e.g. stabilisation or improvement, of said therapy on the disorder or suspected disorder. Furthermore, once treatment has been completed, the method of the invention may be periodically repeated in order to monitor for the recurrence of a disease.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

In a further embodiment, the monitoring of more rapid changes due to fast acting therapies may be conducted at shorter intervals of hours or days.

Kits and Panel Tests

The combination of markers described herein may be used to prepare a kit or panel test, in particular for use in the diagnosis of lung cancer and/or monitoring of patients with lung cancer or suspected lung cancer.

Therefore, according to a further aspect of the invention, there is provided a kit comprising reagents to detect the level of H3K27Me3, H3K36Me3 and CEA. The kits described herein may be for use in the diagnosis of lung cancer.

The kit may comprise reagents for one or more additional biomarkers as described herein. For example, in one embodiment, the kit additionally comprises a reagent to detect the level of CRP.

According to a further aspect of the invention there is provided the use of the kit as defined herein to identify a patient in need of treatment for lung cancer.

According to a further aspect of the invention there is provided the use of the kit as defined herein to monitor a patient for progression of lung cancer (e.g. further growth of the tumour, or advancement to a different stage of cancer). Embodiments of this aspect include use to detect disease progression in watchful waiting, active surveillance and monitoring post-surgery or other treatment for relapse.

According to a further aspect of the invention there is provided the use of the kit as defined herein to evaluate the effectiveness of a lung cancer treatment in a patient.

According to a further aspect of the invention there is provided the use of the kit as defined herein to select a treatment for a patient with lung cancer.

In other embodiments the kit may include reagents to detect total nucleosome levels and/or H1-nucleosome levels and/or may include other nucleosome measurements, such as epigenetic features of a nucleosome (e.g. histone H3.1-levels).

Measurement Methods

In one embodiment, the level or concentration of biomarker (i.e. H3K27Me3, H3K36Me3 and CEA, optionally including CRP) detected is compared to a control. It will be clear to those skilled in the art that the control subjects may be selected on a variety of basis which may include, for example, subjects known to be free of the disease or may be subjects with a different disease (for example, for the investigation of differential diagnosis). The “control” may comprise a healthy subject, a non-diseased subject and/or a subject without cancer. The control may also be a subject with a different stage of cancer, e.g. stage I, stage II, stage III or stage IV cancer. Comparison with a control is well known in the field of diagnostics.

It will be understood that it is not necessary to measure healthy/non-diseased controls for comparative purposes on every occasion because once the ‘normal range’ is established it can be used as a benchmark for all subsequent tests. A normal range can be established by obtaining samples from multiple control subjects without cancer and testing for the level of biomarker. Results (i.e. biomarker levels) for subjects suspected to have cancer can then be examined to see if they fall within, or outside of, the respective normal range. Use of a ‘normal range’ is standard practice for the detection of disease.

If a subject is determined to not have cancer, then the invention may still be used for the purposes of monitoring disease progression. For example, if the use comprises a blood, serum or plasma sample from a subject determined not to have cancer, then the biomarker level measurements can be repeated at another time point to establish if the biomarker level has changed.

References to “subject” or “patient” are used interchangeably herein. In one embodiment, the patient is a human patient. In one embodiment, the patient is a (non-human) animal. The use, panels and methods described herein are preferably performed in vitro.

In one embodiment, detection or measurement of the panel (i.e. H3K27Me3, H3K36Me3 and CEA, optionally including CRP) comprises an immunoassay, immunochemical, mass spectroscopy, chromatographic, chromatin immunoprecipitation or biosensor method.

In one embodiment, the detection or measurement comprises an immunoassay. In a preferred embodiment of the invention there is provided a 2-site immunoassay method for nucleosome moieties. In particular, such a method is preferred for the measurement of nucleosomes or nucleosome incorporated epigenetic features in situ employing two anti-nucleosome binding agents or an anti-nucleosome binding agent in combination with an anti-histone modification or anti-histone variant or anti-DNA modification or anti-adducted protein detection binding agent. In another embodiment of the invention, there is provided a 2-site immunoassay employing a labelled anti-nucleosome detection binding agent in combination with an immobilized anti-histone modification or anti-histone variant or anti-DNA modification or anti-adducted protein binding agent.

Detecting or measuring the level of the biomarker(s) may be performed using one or more reagents, such as a suitable binding agent. In one embodiment, the one or more binding agents comprises a ligand or binder specific for the desired biomarker, e.g. H3K27Me3, H3K36Me3 and CEA, or a structural/shape mimic of the biomarker or component part thereof. The term “biomarker” as defined herein includes any single biomarker moiety or a combination of individual biomarker moieties in a biomarker panel.

As described herein, the level of one or more of the histone biomarkers may be normalised against the level of cell free nucleosomes in the sample, i.e. to determine the proportion of cell free nucleosomes containing the histone biomarkers of interest. Therefore, the level of biomarkers H3K27Me3 and H3K36Me3 may be measured as a ratio of the level of cell free nucleosomes or a component thereof in the sample. In particular, H3K27Me3 and H3K36Me3 may be measured as a ratio of the level of cell free nucleosomes containing a histone H3 variant, such as histone H3.1, in the sample.

It will be clear to those skilled in the art that the terms “antibody”, “binder” or “ligand” in regard to any aspect of the invention is not limiting but intended to include any binder capable of binding to particular molecules or entities and that any suitable binder can be used in the method of the invention.

Methods of detecting biomarkers are known in the art. In one embodiment, the reagents comprise one or more ligands or binders. In one embodiment, the ligands or binders of the invention include naturally occurring or chemically synthesised compounds, capable of specific binding to the desired target. A ligand or binder may comprise a peptide, an antibody or a fragment thereof, or a synthetic ligand such as a plastic antibody, or an aptamer or oligonucleotide, capable of specific binding to the desired target. The antibody can be a monoclonal antibody or a fragment thereof. It will be understood that if an antibody fragment is used then it retains the ability to bind the biomarker so that the biomarker may be detected (in accordance with the present invention). A ligand/binder may be labelled with a detectable marker, such as a luminescent, fluorescent, enzyme or radioactive marker; alternatively or additionally a ligand according to the invention may be labelled with an affinity tag, e.g. a biotin, avidin, streptavidin or His (e.g. hexa-His) tag. Alternatively, ligand binding may be determined using a label-free technology for example that of ForteBio Inc.

Diagnostic or monitoring kits (or panels) are provided for performing methods of the invention. Such kits will suitably comprise one or more ligands for detection and/or quantification of the biomarker according to the invention, and/or a biosensor, and/or an array as described herein, optionally together with instructions for use of the kit.

A further aspect of the invention is a kit for detecting the presence of a disease state, comprising a biosensor capable of detecting and/or quantifying one or more of the biomarkers as defined herein. As used herein, the term “biosensor” means anything capable of detecting the presence of the biomarker. Examples of biosensors are described herein. Biosensors may comprise a ligand binder or ligands, as described herein, capable of specific binding to the biomarker. Such biosensors are useful in detecting and/or quantifying a biomarker of the invention.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace. Biosensors to detect one or more biomarkers of the invention include acoustic, plasmon resonance, holographic, Bio-Layer Interferometry (BLI) and microengineered sensors. Imprinted recognition elements, thin film transistor technology, magnetic acoustic resonator devices and other novel acousto-electrical systems may be employed in biosensors for detection of the one or more biomarkers of the invention.

Biomarkers for detecting the presence of a disease are essential targets for discovery of novel targets and drug molecules that retard or halt progression of the disorder. As the result for a biomarker or biomarker panel is indicative of disorder and of drug response, the biomarker is useful for identification of novel therapeutic compounds in in vitro and/or in vivo assays. Biomarkers and biomarker panels of the invention can be employed in methods for screening for compounds that modulate the activity of the biomarker.

Thus, in a further aspect of the invention, there is provided the use of a binder or ligand, as described, which can be a peptide, antibody or fragment thereof or aptamer or oligonucleotide directed to a biomarker according to the invention; or the use of a biosensor, or an array, or a kit according to the invention, to identify a substance capable of promoting and/or of suppressing the generation of the biomarker.

The term “biomarker” means a distinctive biological or biologically derived indicator of a process, event, or condition. Biomarkers can be used in methods of diagnosis, e.g. clinical screening, and prognosis assessment and in monitoring the results of therapy, identifying subjects most likely to respond to a particular therapeutic treatment, drug screening and development. Biomarkers and uses thereof are valuable for identification of new drug treatments and for discovery of new targets for drug treatment.

The term “detecting” or “diagnosing” as used herein encompasses identification, confirmation, and/or characterisation of a disease state. Methods of detecting, monitoring and of diagnosis according to the invention are useful to confirm the existence of a disease, to monitor development of the disease by assessing onset and progression, or to assess amelioration or regression of the disease. Methods of detecting, monitoring and of diagnosis are also useful in methods for assessment of clinical screening, prognosis, choice of therapy, evaluation of therapeutic benefit, i.e. for drug screening and drug development.

Identifying and/or quantifying can be performed by any method suitable to identify the presence and/or amount of a specific protein in a biological sample from a subject or a purification or extract of a biological sample or a dilution thereof. In methods of the invention, quantifying may be performed by measuring the concentration of the target in the sample or samples. Biological samples that may be tested in a method of the invention include those as defined hereinbefore. The samples can be prepared, for example where appropriate diluted or concentrated, and stored in the usual manner.

Identification and/or quantification of biomarkers may be performed by detection of the biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length. It is noted in particular that peptides of the same or related sequence to that of histone tails are particularly useful fragments of histone proteins.

For example, detecting and/or quantifying can be performed using an immunological method, such as an immunoassay. Immunoassays include any method employing one or more antibodies or other specific binders directed to bind to the biomarkers defined herein. Immunoassays include 2-site immunoassays or immunometric assays employing enzyme detection methods (for example ELISA), fluorescence labelled immunometric assays, time-resolved fluorescence labelled immunometric assays, chemiluminescent immunometric assays, immunoturbidimetric assays, particulate labelled immunometric assays and immunoradiometric assays as well as single-site immunoassays, reagent limited immunoassays, competitive immunoassay methods including labelled antigen and labelled antibody single antibody immunoassay methods with a variety of label types including radioactive, enzyme, fluorescent, time-resolved fluorescent and particulate labels.

In another example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods involving identification and/or quantification of one or more biomarkers of the invention can be performed on bench-top instruments, or can be incorporated onto disposable, diagnostic or monitoring platforms that can be used in a non-laboratory environment, e.g. in the physician's office or at the subject's bedside. Suitable biosensors for performing methods of the invention include “credit” cards with optical or acoustic readers. Biosensors can be configured to allow the data collected to be electronically transmitted to the physician for interpretation and thus can form the basis for e-medicine.

The identification of biomarkers for a disease state permits integration of diagnostic procedures and therapeutic regimes. Detection of a biomarker of the invention can be used to screen subjects prior to their participation in clinical trials. The biomarkers provide the means to indicate therapeutic response, failure to respond, unfavourable side-effect profile, degree of medication compliance and achievement of adequate serum drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, subject care can be tailored precisely to match the needs determined by the disorder and the pharmacogenomic profile of the subject, the biomarker can thus be used to titrate the optimal dose, predict a positive therapeutic response and identify those subjects at high risk of severe side effects.

Biomarker-based tests provide a first line assessment of ‘new’ subjects, and provide objective measures for accurate and rapid diagnosis, not achievable using the current measures.

Biomarker monitoring methods, biosensors and kits are also vital as subject monitoring tools, to enable the physician to determine whether relapse is due to worsening of the disorder. If pharmacological treatment is assessed to be inadequate, then therapy can be reinstated or increased; a change in therapy can be given if appropriate. As the biomarkers are sensitive to the state of the disorder, they provide an indication of the impact of drug therapy.

It will be understood that the embodiments described herein may be applied to all aspects of the invention, i.e. the embodiment described for the uses may equally apply to the claimed methods and so forth.

The invention will now be illustrated with reference to the following non-limiting examples.

### EXAMPLES

### Example 1

EDTA plasma samples were obtained from 220 patients previously scanned by LDCT for lung cancer including 70 patients in whom no nodules were found, 100 patients in whom nodules were found and confirmed as malignant by histology of biopsy tissue samples removed surgically and 50 patients in whom nodules were found and confirmed as non-malignant by histology of biopsy tissue samples removed surgically. The details of the patient cohort are outlined in Table 1.

TABLE 1
Demographics of the study cohort

| | | | | | Family |
|---|---|---|---|---|---|
| | | Age | | | History:No |
| | Patients | (median, | Sex | Smoker:Non- | Family |
| Diagnosis | (n) | IQR) | (M:F) | smoker | History |
| Lung Cancer | 100 | 62 (53-70) | 39:61 | 20:80 | 32:68 |
| Stage 0-I | 67 | 60 (50-67) | 16:51 | 4:63 | 23:44 |
| Stage II | 3 | 66 (52-68) | 2:1 | 1:2 | 1:2 |
| Stage III | 15 | 69 (55-73) | 10:5 | 9:6 | 5:10 |
| Stage IV | 15 | 66 (55-71) | 11:4 | 6:9 | 3:12 |
| Non- | 50 | 58 (50-66) | 20:30 | 9:41 | 15:35 |
| malignant | | | | | |
| Nodules | | | | | |
| Healthy | 70 | 57 (45-66) | 23:47 | 12:58 | 32:38 |

Whole blood samples were collected into EDTA plasma tubes and tested against chosen biomarkers. Levels of CEA and CRP were measured using commercially available immunoassay methods. H3K27Me3 and H3K36Me3 were also measured using a sandwich immunoassay method employing one antibody directed to bind to the selected histone modification and one antibody directed to bind to an epitope present in intact nucleosomes.

Assay results were modelled by Logistic Regression analysis to train for the model or algorithm with the highest AUC for a comparison of patients with lung cancer vs patients with non-malignant nodules (benign). For preparing an appropriate diagnostic test, it is recommended that the most specific test is used to confirm (i.e. rule-in) a diagnosis and the most sensitive test is used to establish that a disease is unlikely (i.e. rule-out). The results are summarised in Table 2.

TABLE 2
Summary of results

| | | Rule-in | | Rule-out | |
|---|---|---|---|---|---|
| | | Sensi- | Sensi- | Speci- | Speci- |
| | | tivity | tivity | ficity | ficity |
| | | at 95% | at 100% | at 95% | at 100% |
| | | Speci- | Speci- | Sensi- | Sensi- |
| Model | AUC | ficity | ficity | tivity | tivity |

Whole Cohort

| | | | | | |
|---|---|---|---|---|---|
| LnY = H3K27Me3 + | 0.742* | 42% | 33% | 14% | 4.7% |
| H3K36Me3 + CRP + | | | | | |
| CEA | | | | | |
| LnY = H3K27Me3 + | 0.730* | 27.3% | 26.1% | 18.6% | 4.7% |
| H3K36Me3 + CEA | | | | | |

Smoker only

| | | | | | |
|---|---|---|---|---|---|
| LnY = H3K27Me3 + | 0.819* | 66.7% | 66.7% | 12.5% | 12.5% |
| H3K36Me3 + CRP + | | | | | |
| CEA | | | | | |

No Family Cancer History

| | | | | | |
|---|---|---|---|---|---|
| LnY = H3K27Me3 + | 0.795* | 45.9% | 34.4% | 32.3% | 16.1% |
| H3K36Me3 + CRP + | | | | | |
| CEA | | | | | |
| LnY = H3K27Me3 + | 0.797* | 27.9% | 26.2% | 45.2% | 22.6% |
| H3K36Me3 + CEA | | | | | |

No Family Cancer History and Early Stage Lung Cancer

| | | | | | |
|---|---|---|---|---|---|
| LnY = H3K27Me3 + | 0.777 | 32.5% | 17.5% | 35.5% | 22.6% |
| H3K36Me3 + CRP + | | | | | |
| CEA | | | | | |
| LnY = H3K27Me3 + | 0.772 | 10% | 7.5% | 54.8% | 32.3% |
| H3K36Me3 + CEA | | | | | |

*AUC is significantly different from chance.

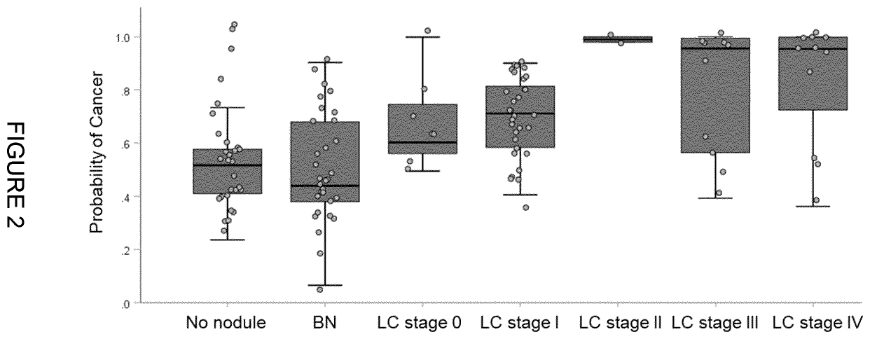

The panels tested were able to provide suitable rule-in and rule-out tests for lung cancer. In particular, the panel comprising H3K27Me3+H3K36Me3+CEA was able to rule-in cancer in 27% of patients with cancerous nodules among the whole cohort with high (95%) specificity. (example results presented in FIG. 2). Therefore the panel is useful to confirm more than a quarter of all cancer cases in need of further investigation and treatment.

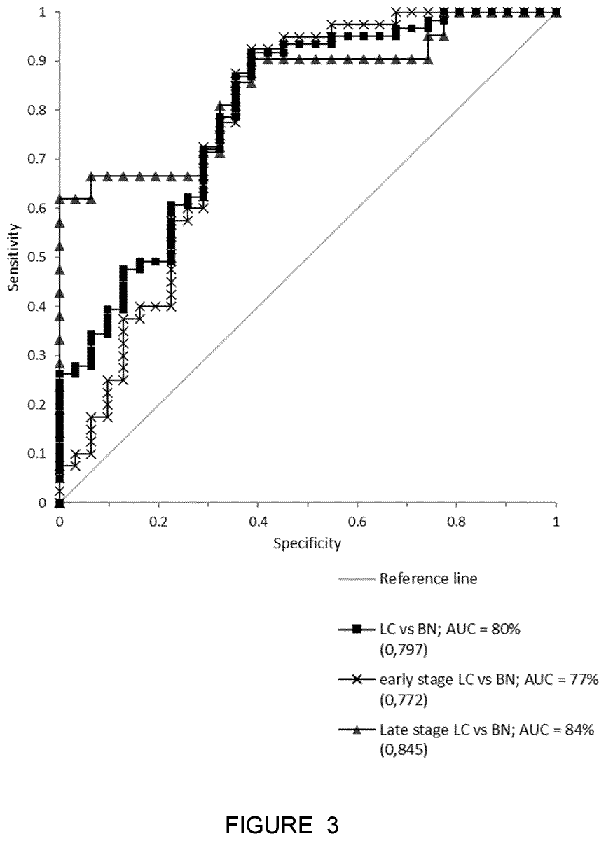

The panel worked particularly well in patients with no family history of lung cancer and was able to rule-out 32% of patients with non-malignant nodules as not having an early stage cancer (0, I and II) with 100% specificity (Receiver Operating Characteristic (ROC) curves presented in FIG. 3). Early stage cancers are more likely to present with smaller nodules therefore a rule-out test for early stage cancers is highly useful in the context of screening programs because scanning methods such as LDCT have poor specificity for differentiation of non-malignant nodules leading to unnecessary biopsy or repeat scans. Thus the results of other clinical parameters may be used with the panel to further increase accuracy and utility.

Addition of CRP to the panel provided a useful detection (or rule-in) method for lung cancer, particularly in smokers (with and without a family history of lung cancer). The panel was able to detect 42% of cancers at 95% specificity among all the subjects tested. This was increased to 66.7% of cancers at 95% specificity in patients who were smokers (a population often targeted in lung cancer screening programs).

### Example 2

We hypothesised that the proportion of nucleosomes present in the circulation that contain a particular histone modification may be more clinically relevant than the simple amount of those nucleosomes present. We tested this hypothesis on a large cohort of patients screened by LDCT.

EDTA plasma samples were obtained from 702 patients previously scanned by LDCT for lung cancer including 77 patients in whom no nodules were found and 625 patients in whom nodules were found and confirmed as malignant by histology of biopsy tissue samples removed surgically. Of the 625 patients with a confirmed diagnosis of lung cancer, 55 had stage 0 disease, 397 stage I disease, 35 stage II disease, 43 stage III disease, 68 stage IV disease and 27 had lung cancer of unknown stage. Therefore, more than 70% of patients had early stage 0 or stage I disease.

Levels of CEA were measured using a commercially available immunoassay method. Nucleosomes containing histone variant H3.1 as well as histone modifications H3K27Me3 and H3K36Me3 were measured using a sandwich immunoassay method employing one antibody directed to bind to the selected histone variant or modification and one antibody directed to bind to an epitope present in intact nucleosomes.

We calculated ROC curves and AUCs for the discrimination of subjects with and without cancer for each assay using Logistic Regression analysis. We also calculated ROC curves and AUCs for the ratios of H3K27Me3/H3.1 and H3K36Me3/H3.1, as well as for panels including the ratios as shown in Table 3.

TABLE 3
AUCs calculated for assay results, nucleosome ratio results
and panels for the 625 patient cohort (and for patients with
each disease stage 0, I, II, III, IV within the cohort)

| Assay, ratio | AUC |
|---|---|
| or panel | (AUC stage 0, I, II, III, IV) |
| CEA | 65% (43, 59, 81, 83, 94%) |
| H3.1 | 56% (51, 52, 61, 65, 69%) |
| H3K27Me3 | 50% (47, 47, 56, 53, 63%) |
| H3K36Me3 | 49% (45, 46, 54, 55, 63%) |
| H3K27Me3/H3.1 | 68% (63, 66, 73, 70, 77%) |
| H3K36Me3/H3.1 | 64% (61, 64, 65, 64, 61%) |
| H3K27Me3/H3.1 + H3K36Me3/H3.1 | 71% (65, 69, 74, 73, 76%) |
| H3K27Me3/H3.1 + CEA | 70% (54, 64, 85, 87, 94%) |
| H3K36Me3/H3.1 + CEA | 71% (55, 66, 85, 86, 95%) |
| *CEA // H3K27Me3/ | 76% (64, 72, 89, 86, 93%) |
| H3.1 + H3K36Me3/H3.1 | |

*Denotes a decision tree analysis where patients were classified as positive for cancer if either the CEA level was abnormal (>5 ng/ml) or if the logistic regression result for H3K27Me3/H3.1 + H3K36Me3/H3.1 was abnormal (or both).

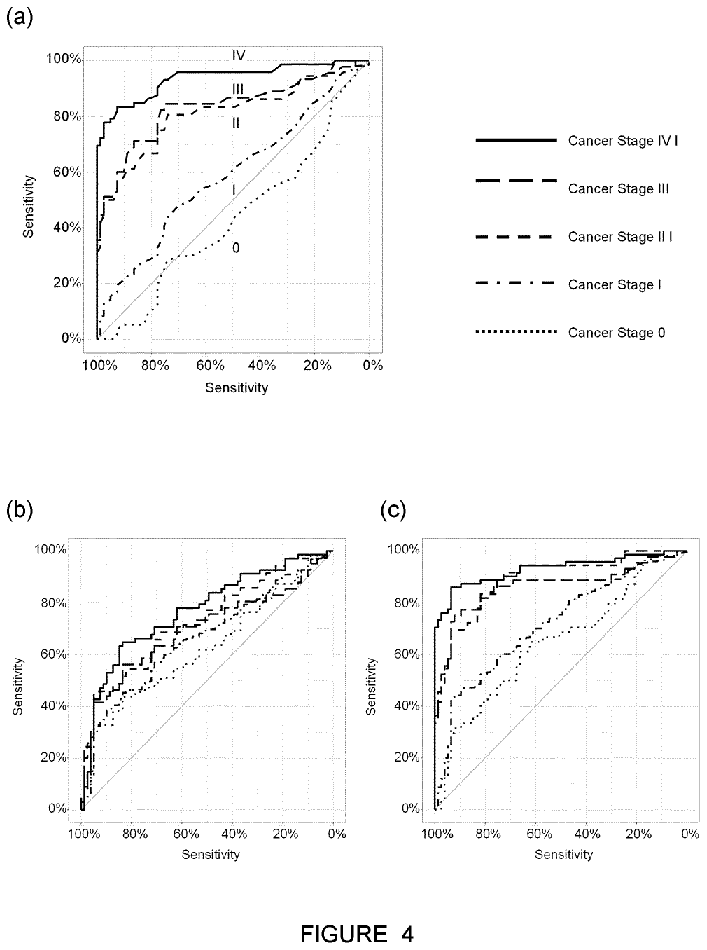

ROC curves for the detection of stage 0, I, II, Ill and IV lung cancer for CEA, H3K27Me3/H3.1 and the decision tree analysis CEA//H3K27Me3/H3.1+H3K36Me3/H3.1 are also shown graphically in FIG. 4.

The results in Table 3 and FIG. 4 show that CEA is a disease stage dependent marker for lung cancer (overall cohort AUC=65%) and is a good marker for stage II and III lung cancer and an excellent marker for stage IV cancer. However, it is less good for stage 0 or stage I. The individual H3.1, H3K27Me3 and H3K36Me3 nucleosome levels are also stage dependent markers but are individually less discriminating than CEA (overall cohort AUCs=56 and 50%). This indicates that the levels are reflective of disease and disease severity.

However, we observed that normalising the individual histone modification levels H3K27Me3 and H3K36Me3 as ratios to the level of H3.1 nucleosomes (i.e. H3K27Me3/H3.1 and H3K36Me3/H3.1) improved the discrimination for cancer to similar or better levels to that of CEA (overall cohort AUCs=68 and 64%). The ratio markers are also stage dependent and reflective of disease and disease severity. Moreover the ratios have improved discrimination for stage 0 and stage 1 cancer.

A combination of the two ratios using Logistic Regression analysis or a combination of either ratio with CEA improved the results further (overall cohort AUCs=70, 71 and 70%).

A combination of CEA and the two ratios in a decision tree analysis wherein patients were classified as positive for cancer if either the CEA level was abnormal (>5 ng/ml) or if the logistic regression result for H3K27Me3/H3.1+H3K36Me3/H3.1 was abnormal (or both), further improved the discrimination for cancer (overall cohort AUC=76%) with much improved discrimination for early stage cancer compared to CEA.

### REFERENCES

Bjorkman et al, Scandinavian J Immunol, 57: 525-533, 2003
Chadha et al, Clin Cancer Investig J, 3: 72-79, 2014
Herranz and Esteller, Methods Mol Biol, 361: 25-62, 2007
Holdenrieder et al, Int J Cancer, 95: 114-20, 2001
Holdenrieder et al, Clin Chem, 51(6): 1026-1029, 2005
Holdenrieder and Stieber, Crit Rev Clin Lab Sci, 46(1): 1-24, 2009
Lazris et al, Am Fam Physician. 99(12): 740-742, 2019
Midthun, F1000Res, 5: F1000 Faculty Rev-739, 2016
Moyer, Ann Intern Med, 160(5): 330-338, 2014
Salgame et al, Nucleic Acids Res, 25(3): 680-681, 1997
van Nieuwenhuijze et al, Ann Rheum Dis, 62: 10-14, 2003
Zhao and Garcia, Cold Spring Harb Perspect Biol, 7: a025064, 2015

1. A method of diagnosing or monitoring lung cancer in a patient, comprising:
detecting or measuring a panel of biomarker levels in a body fluid sample, wherein the panel of biomarkers comprises H3K27Me3, H3K36Me3 and Carcinoembryonic Antigen (CEA); and
using the levels detected in the body fluid sample to diagnosis lung cancer or to monitor lung cancer.

2. The method of claim 1, wherein the panel comprises one or more additional biomarkers.

3. The method of claim 2, wherein the additional biomarker is C-Reactive Protein (CRP).

4. The method of claim 1, wherein the body fluid sample is a blood, serum or plasma sample.

5. The method of claim 1, wherein the lung cancer is early-stage lung cancer.

6. The method of claim 1, wherein H3K27Me3 and H3K36Me3 are measured as a ratio of the level of cell-free nucleosomes or a component thereof in the body fluid sample.

7. The use as defined in method of claim 6, wherein H3K27Me3 and H3K36Me3 are measured as a ratio of the level of cell-free nucleosomes containing histone H3.1 in the body fluid sample.

8. (canceled)

9. The method of claim 1,
wherein said using comprises using the level detected in the body fluid sample to determine if the patient requires further testing for lung cancer.

10. The method of claim 9, wherein the further testing for lung cancer is a lung biopsy.

11. The method of claim 1, wherein the lung cancer is early-stage lung cancer.

12. The method of claim 1, wherein the patient has a pulmonary nodule.

13. The method of claim 1, wherein the level of H3K27Me3 and H3K36Me3 are measured as a ratio of the level of cell-free nucleosomes or a component thereof in the body fluid sample.

14. The method of claim 13, wherein the level of H3K27Me3 and H3K36Me3 are measured as a ratio of the level of cell-free nucleosomes containing histone H3.1 in the body fluid sample.

15. The method of claim 1 wherein said detecting or measuring comprises detecting or measuring a level of C-Reactive Protein (CRP).

16. The method of claim 1, further comprising determining at least one clinical parameter for the patient.

17. The method of in claim 16, wherein the clinical parameter is selected from the group consisting of smoking status and family history of lung cancer.

18. The method of claim 1, wherein the level of H3K27Me3, H3K36Me3 and CEA detected is compared to a control.

19. (canceled)

20. The method of claim 1, wherein the detecting or measuring is performed using an immunoassay, immunochemical, mass spectroscopy, chromatographic, chromatin immunoprecipitation or biosensor method.

21. A method of treating lung cancer in a patient, comprising;
(i) detecting or measuring levels of H3K27Me3, H3K36Me3 and CEA, in a body fluid sample obtained from the patient;
(ii) using the levels detected or measured in the body fluid sample to determine if the patient has lung cancer; and
(iii) administering a treatment to the patient if the patient is determined to have lung cancer in step (ii).

22. A kit comprising reagents to detect H3K27Me3, H3K36Me3 and CEA.

23. (canceled)

24. (canceled)
